(19) United States
(12) Patent Application Publication
(10) Pub. No.: US 2002/0173446 A1
(43) Pub. Date: Nov. 21, 2002

(54) Compounds which bind to the central cavity between HIV-1 gp120 and CD4 and uses thereof

(76) Inventors: Peter Kwong, New York, NY (US); Wayne Hendrickson, New York, NY (US); Richard Wyatt, Andover, MA (US); Joseph Sodroski, Medford, MA (US)

Correspondence Address: John P. White, Esq., Cooper & Dunham, LLP, 1185 Avenue of the Americas, New York, NY, 10036, US

(21) Appl. No.: 10/012,507

(22) Filed: Dec. 7, 2001

Related U.S. Application Data
(60) Provisional application No. 60/254,046, filed on Dec. 7, 2000.

(51) Int. Cl. A61K 31/00; C12Q 1/70

(52) U.S. Cl. 514/1; 435/5; 702/19

(57) ABSTRACT

This invention provides a method for identifying a compound which inhibits HIV-1 entry into a cell. This invention also provides a compound which inhibits the cavity binding interaction between HIV-1 gp120 and CD4. This invention further provides a method of inhibiting HIV-1 infection of a cell, a method of preventing HIV-1 infection in a subject and a method of treating HIV-1 infection in a subject comprising contacting the cell or administering to the subject an amount of the compound which inhibits the cavity binding interaction between HIV-1 gp120 and CD4 effective to inhibit HIV-1 infection, thereby inhibiting HIV-1 infection of the cell, preventing HIV-1 infection in the subject and treating HIV-1 infection in the subject.

This application is a continuation-in-part of U.S. Provisional Application No. 60/254,046, filed Dec. 7, 2000, the contents of which are hereby incorporated by reference.

The invention disclosed was herein made in the course of work under NIH Grant No. AI 31783, NIH Grant No. AI 39420, and a Cancer Center Grant (CA 06516) from the National Institutes of Health. Accordingly, the U.S. Government has certain rights in this invention.

Throughout this application, various references are referred to within parentheses. Disclosures of these publications in their entireties are hereby incorporated by reference into this application to more fully describe the state of the art to which this invention pertains. Full bibliographic citation for these references may be found at the end of this application, preceding the claims.

### BACKGROUND OF THE INVENTION

The human immunodeficiency virus (HIV-1) gp120 exterior envelope glycoprotein binds sequentially to the CD4 receptor and a chemokine receptor on the cell surface to initiate virus entry. During natural infection, gp120 is a primary target of the humoral immune response and thus has evolved to resist antibody-mediated neutralization. We previously reported the structure at 2.5 Å of a gp120 core from the HXBc2 laboratory-adapted isolate in complex with a two-domain fragment of CD4 and the antigen-binding fragment of a human antibody. This structure revealed details of the gp120-receptor interaction as well as multiple potential mechanisms of immune evasion.

We have extended the refinement of the previously reported HXBc2 P222₁ crystals to 2.2 Å. The enhanced resolution, corresponding to a 150% increase in data, has permitted the conclusive identification of the density in the central cavity at the crux of the gp120-CD4 interaction as isopropanol, which was present in the crystallization medium. It has also allowed more accurate modeling of less well-ordered regions. We have also determined the structure of a gp120 core from the primary clinical HIV-1 isolate, YU2, in the same ternary complex, but a C2 crystal lattice. Comparisons of HXBc2 and YU2 showed that while CD4 binding was rigid, portions of the gp120 core were conformationally flexible; overall differences were minor, with sequence changes concentrated in a region that is expected to be exposed on the surface of the envelope oligomer.

Given the dramatic antigenic differences between primary and laboratory-adapted HIV-1, the gp120 cores from these isolates are remarkably similar. Thus changes specifying neutralization resistance must be limited to the major variable loops or sites of oligomer interaction, or to subtle alterations in the cores within the level of the differences between crystal lattices. The refined structures also reveal additional features of the gp120-receptor interaction: flexibility in the chemokine receptor binding site, atomic details of the CD4 interaction and molecular breathing of the complex. Conservation of the central cavity suggests the possibility of therapeutic inhibitors, analogous to the pocket-binding compounds effective against poliovirus and rhinovirus. The structures reported here extend in detail and generality our understanding of the biology of the gp120 envelope glycoprotein.

The structures disclosed herein further provide methods of determining drugs, i.e. compounds that bind to the central cavity, i.e. pocket, between HIV-1 gp120 and CD4, and thus, prevent entry of HIV-1 into cells. Methods include, but are not limited to, determining which compounds “soak” into the cavity and tightly bind so as to block HIV-1 entry into cells. Compounds determined by these methods are analogous to the pocket-binding compounds effective against poliovirus and rhinovirus. (e.g. [Grant et al. (1994); Joseph-McCarthy et al. (1997)] The compounds determined by these methods are useful as therapeutic agents for HIV-1 infection and AIDS, therefore, the structure of the pocket between gp120 envelope glycoprotein and CD4 provided herein is useful for designing drugs which tightly bind to the cavity between HIV-1 gp120 and CD4, since the cavity is highly conserved. In addition to chemical compounds which tightly bind to the cavity between HIV-1 gp120 and CD4, the subject invention provides methods of mutating gp120 to fill the cavity, so as to alter CD4 binding, thereby blocking entry of HIV-1 into cells, gp120 mutants such as 375 S/W fill the cavity and thereby reduces HIV-1 infectivity substantially. Other mutants which fill the cavity may thus be designed according to the subject invention. Drugs may also be designed, according to the subject invention, by attaching a compound(s) to CD4, so as to produce such drug as antagonists.

Human immunodeficiency virus (HIV-1) causes the depletion of CD4+ T-lymphocyctes and, eventually, acquired immunodeficiency syndrome (AIDS) in chronically infected humans [Barre-Sinoussi, 1983; Gallo, 1984]. HIV-1 entry into target cells is mediated by the viral envelope glycoproteins, gp120 and gp41, which are organized into trimeric complexes on the virion surface [Veronese, 1985 1; Robey, 1985; Allan, 1985]. The gp120 exterior envelope glycoprotein binds the CD4 glycoprotein on target cells, triggering conformational changes in gp120 that allow binding to one of two chemokine receptors, CCR5 or CXCR4 [Dalgleish, 1984; Feng, 1996; Deng, 1996; Choe, 1996; Dragic, 1996; Alkhatib, 1996; Doranz, 1996]. Receptor binding is thought to lead to the exposure of the ectodomain of the gp41 transmembrane envelope glycoprotein and additional conformational changes that result in the fusion of the viral and cell membrane [Chan, 1997; Weissenhorn, 1997]. The exposed location of the gp120 glycoprotein on the virus, which is necessitated by the requirement for receptor binding, renders the protein potentially vulnerable to neutralizing antibodies. Several of the characteristics of the HIV-1 gp120 glycoprotein, a high level of glycosylation, the presence of surface-exposed variable loops (V1-V5), and conformational flexibility, are thought to have evolved to decrease the susceptibility of the virus to the host humoral immune response [Kwong, 1998; Myszka, 2000; Wyatt, 1998; Wyatt, 1998].

Elements of gp120 that are relatively well conserved among HIV-1 isolates fold into a “core”, which has been crystallized in a complex with the amino-terminal two domains (D1D2) of CD4 and the antigen-binding fragment (Fab) of the human neutralizing antibody, 17b [Kwong, 1998]. The gp120 core is composed of an inner and outer domain, reflecting the likely orientation of gp120 in the assembled trimer, and a bridging sheet. Components of both domains and the bridging sheet contribute to CD4 binding. CD4 binds in a recessed pocket on gp120 to a surface that is larger than that occluded by a typical antibody-protein interaction. The interface displays several unusual features, including a shallow water-filled cavity, which is thought to function in immune evasion and is also seen in the adenovirus virus: CAR receptor complex [Bewley, 1999]. A second interfacial cavity penetrates into the heart of gp120, and is bounded by conserved interior gp120 residues derived from all three domains, and by phenylalanine 43 (Phe 43) of CD4. Mutagensis, conservation and structural analysis all indicate that this “Phe 43 cavity” and its surrounding structures are critically important for CD4 binding. The Phe 43 cavity constitutes a conserved, spacially localized feature in a large otherwise relatively variable gp120-CD4 interface. As such, it appears to be a potential target for small molecular weight antagonists of gp120-CD4 binding.

The chemokine receptor-binding surface of gp120 is thought to be composed of variable and conserved elements and to be oriented towards the target cell membrane by CD4 binding [Kwong, 1998]. The third variable (V3) loop sequence determines the choice of chemokine receptor [Choe, 1996; Cocchi, 1996; Bieniasz, 1997; Speck, 1997]. In addition, a highly conserved gp120 structure near the bridging sheet has been shown to undergo conformational changes upon CD4 binding, to contain residues important for CCR5 binding, and to serve as a target for neutralizing antibodies that block chemokine receptor binding [Rizzuto, 1998]. Presumably, this conserved basic surface of gp120 cooperates with the V3 loop to create a binding site for the relatively acidic CCR5 ectodomains [Farzan, 1999].

The persistence of HIV-1 infections necessitates that conserved gp120 structures involved in receptor binding are poorly immunogenic and/or exhibit limited accessibility to potentially neutralizing antibodies. HIV-1 viruses that have been passaged in immortalized cell lines are typically more sensitive to antibody neutralization than are primary clinical isolates [Mascola, 1994; Wrin, 1995; Sawyer, 1994; Sullivan, 1995]. The most important determinants of this resistance to neutralization are the major gp120 variable loop, V1/V2 and/or V3 [Koito, 1994; Hwang, 1991; Morikita, 1997; Sullivan, 1998]. In several cases, N-linked carbohydrate on or near these variable loops influences the sensitivity of primary HIV-1 isolates to neutralization by antibodies [Schonning, 1996; Ly, 2000]. The basis for the decreased sensitivity of primary isolates to neutralization appears to be a decreased exposure of the relevant epitopes in the context of the assembled, trimeric HIV-1 envelope glycoprotein complex [Sullivan, 1998; Fouts, 1997; Parren, 1999].

Additional structures of the HIV-1 envelope glycoproteins derived from primary, clinical HIV-1 isolates, either free or complexed with receptors or neutralizing antibodies, will provide insights useful in the quest for HIV-1 therapeutics and vaccines. Here we report the refined structure at 2.2 Å of a gp120 core derived from a laboratory-adapted HIV-1 isolate, HXBc2, in a complex with D1D2 of CD4 and the Fab fragment of the 17b antibody. We also report the structure at 2.9 Å of a gp120 core derived from a primary clinical HIV-1 isolate, in the same ternary complex. This primary isolate, YU2, had not been propagated in tissue culture prior to molecular cloning [Li, 1991]. Thus several aspects of the gp120 core structure, receptor binding, and interactions with neutralizing antibodies can be compared for laboratory-adapted and primary HIV-1.

### SUMMARY OF THE INVENTION

This invention provides a method for identifying a compound which inhibits HIV-1 entry into a cell:(a) determining the conformation of a crystal of HIV-1 gp120 in the presence of a ligand; (b) contacting the crystal of HIV-1 gp120 with the compound; (c) determining the conformation of the crystalized HIV-1 gp120 central cavity in the presence of the compound; and (d) comparing the conformation of the crystalized HIV-1 gp120 central cavity determined in step (c) with the conformation of the crystalized HIV-1 gp120 in step (a), so as to identify a compound which inhibits HIV-1 entry into the cell.

This invention provides a compound which inhibits the cavity binding interaction between HIV-1 gp120 and CD4.

This invention provides a method of inhibiting HIV-1 infection of a cell comprising contacting the cell with an amount of the compound which inhibits the cavity binding interaction between HIV-1 gp120 and CD4 effective to inhibit HIV-1 infection, thereby inhibiting HIV-1 infection of the cell.

This invention provides a method of preventing HIV-1 infection in a subject comprising administering to the subject an amount of the compound which inhibits the cavity binding interaction between HIV-1 gp120 and CD4 effective to inhibit HIV-1 infection, thereby preventing HIV-1 infection in the subject.

This invention provides a method of treating HIV-1 infection in a subject comprising administering to the subject an amount of the compound which inhibits the cavity binding interaction between HIV-1 gp120 and CD4 effective to inhibit HIV-1 infection; thereby treating HIV-1 infection in the subject.

### BRIEF DESCRIPTION OF THE FIGURES

FIG. 1. Overall structure of HXBc2 and YU2 Ternary Complexes. The HXBc2 ternary complex is shown in Ca worm representation for the gp120 core (red), the N-terminal two domains of CD4 (yellow) and the Fab portion of the 17b neutralizing antibody (blue). The YU2 gp120 core has been superimposed on the HXBc2 core, and a Cα worm (green) is shown for the CD4 and 17b portions of the YU2 complex. For the YU2 core, the molecular surface of all amino acids that differ between the two isolates has been colored green. The gp120 cores are oriented around a vertical trimer axis, as defined by the optimization of quantifiable surface parameters [Kwong, 2000]. Thus, the virus membrane is positioned at the top of the picture and the target cell membrane at the bottom. Mutually perpendicular views of gp120 are shown in FIGS. 2a/2b and FIG. 4. The figure was made with GRASP [Nicholls, 1991].

FIGS. 2a-2b. Structure of HXBc2 and YU2 gp120. In (2a) and (2b), the orientation of gp120 is related to FIG. 1 by a 90 rotation about a vertical axis, and to the right-most molecule in FIG. 4 by a 90° rotation about a horizontal axis. In the orientation shown, the viral membrane is positioned above the molecule, the target cell membrane below. (2a) Ribbon diagram. In the left panel, the HXBc2 core is depicted in red (α-helices) and salmon (β-stands) except for β15 in yellow, which hydrogen bonds to the C″ strand of CD4. The inner domain (N terminus-α1, β4-β8, and α5-C terminus) bridging sheet (β2, β3, β20, and β21), and outer domain (β9-β19 and β22-β24) are labeled. In the right panel, the YU2 core is depicted in green (α helices) and light green (β strands) except for β15. The N terminus (N) and C terminus (C) are labeled, as are the sequence-variable loops. (2b) Stereoplot of the Cα superposition of HXBc2 (red) and YU2 (black) core gp120. Every 10^{th} Cα is marked with a filled circle, and every 20th residue of YU2 is labeled. Disulfide connections are depicted in ball-and-stick representations. Only ordered residues are drawn. The figure was drawn with MOLSCRIPT [Kraulis, P. J. et al. (1991)]

FIG. 3. Sequence of HXBc2 and YU2 gp120. The HXBc2 and YU2 core sequences are shown along with labeled secondary-structural assignments, as follows: cylinders for helices, arrows for strands, and “X” for disorder. The lowercase “gars” and “gag” sequences are artifacts of the expression system and loop truncation, respectively. The atomic mobility (B) factors are shown for HXBc2 and YU2. The numbers shown represent the B factors divided by ten and rounded to the nearest integer. B factors of less than 15 and greater than 85 Å² were assigned values of 1 and 9, respectively. The root-mean-square deviations (rmsd) for YU2 and HXBc2 after C∝superposition are shown rounded to the nearest Å. Sequence identities and rmsd of less than 0.5 Å are depicted with a period; sequence gaps and rmsd for nonconserved residues are depicted with a dash. Asterisks denote changes in N-linked glycosylation. Although truncated in the core, the V3 loop sequence is also shown for residues 296-331. Residues have been numbered according to the HXBc2 gene sequence, with the mature full-length protein beginning at residue 31.

FIG. 4. HXBc2 and YU2 Sequence Differences in the Context of the Modeled Envelope Oligomer The gp120 core is depicted as a trimer, as oriented by optimization of quantifiable surface parameters [39]. The C∝ worm of HXBc2 is shown in red. The YU2 core has been superimposed onto the HXBc2 core, and the molecular surface of any sequence differences is depicted in green. The (N-acetylglucosamine) 2 (mannose) 3 cores (modeled as in [17]) of the carbohydrate common to both HXBc2 and YU2 are shown in cyan, those specific to HXBc2 are shown in red, and those specific to YU2 are shown in green. The view shown is from the perspective of the target cell membrane. Mutually perpendicular orientations are shown in FIGS. 1 and 2. The figure was created with GRASP [72].

FIG. 5. The Central Phe-43 Cavity between CD4 and gp120 Different portions of this figure all show the Phe-43 cavity from the same orientation. (5a) C∝ worm diagram of the YU2 core (green) binding to CD4 (yellow). The critical Phe-43 side chain is seen reaching into the heart of gp120. The molecular surface of the Phe-43 cavity at the gp120-CD4 interface is colored blue. (5b) Electron density of the Phe-43 cavity. The 2F₀-F_{c} electron density is depicted at 1.1σ contour (blue). The 2.2 Å HXBc2 structure is shown in the left panel; the 2.9 Å YU2 structure is shown in the right panel. The HXBc2 core is colored red; the YU2 core, green; the CD4, yellow; and the water molecules, cyan. An isopropanol is shown at the center of the HXBc2 cavity. It is colored yellow for carbon atoms and red for its hydroxyl atom. CD4 residues are labeled in yellow, and YU2 residues are labeled in green. (5c) Stereoplot of the HXBc2 Phe-43 cavity. The isopropanol is colored red. Hydrogen bonds of the isopropanol hydroxyl to neighboring water molecules (and their respective water-specific hydrogen bonds) are depicted with dotted blue lines. Panel (5a) was drawn with GRASP [72]; panels (5b) and (5c) were drawn with 0 [74].

FIG. 6. Domain Flexibility in HXBc2 and YU2 Ternary Complexes. Individual domains (specified by the left-most column) of the HXBc2 ternary complex structure were superimposed on the corresponding domains in the YU2 structure. After each superposition, the root-mean-square deviation (rmsd) in C_{α} position was calculated for individual domains (specified in the top row). The bold numbers along the table diagonal represent the rmsd of the optimal superposition of each domain. They represent an estimate of the internal domain flexibility. For gp120, all residues conserved and ordered between HXBc2 and YU2 were superimposed. For the gp120 core, the following 266 C_{α} positions were analyzed: resides 83-118, 204-256, and 474-492 of the inner domain (101 positions); residues 257-421 and 436-473 of the outer domain (131 positions); and residues 119-203 and 422-435 of the bridging sheet (34 positions). For CD4 and 17b, only the domains closest to gp120 were used; for CD4, residues 1-98 were used; and for 17b, residues 1-109 of the light chain and 1-127 of the heavy chain were used.

FIG. 7. CD4 Conformational Conservation. Five different CD4 molecules were used. CD4-1 refers to the 2.3 Å C2 structure of the 2 domain CD4 molecule with the cell constants a=83.71 Å, b=30.07 Å, c=87.54 Å, and β=117.28° (PDB accession code 1cdh [43]). CD4-2 refers to the 2.9 Å C2 structure of the 2 domain CD4 molecule with the cell constants a=133.44 Å, b=32.07 Å, c=45.85 Å, and β=96-18° (PDB accession code 1cdi[43]). CD4-3 refers to the 3.9 A P4₃22 structure of the 4 domain CD4 molecule [451. The HXBc2 and YU2 complexes are the 2 domain CD4 complexes with gp120 reported here. Each N-terminal domain (domain 1; residues 1-98) specified in the left-most column was superimposed on the N-terminal domain of a different CD4 structure specified in the top row, and the rmsd was calculated. A second superposition of the second domain of CD4 (domain 2; residues 99-178) was performed, and both the rotational angle of superposition (the angular deviation of the interdomain separation) and rmsd were determined.

FIG. 8. Structure Solution and Refinement Statistics.

^{a}R_{sym}=Σ|/_{obs}−/_{avg}|Σ/_{avg}.

^{b}Numbers in parentheses represent the statistics for the shell comprising the outer 10% (theoretical) of the data.

### DETAILED DESCRIPTION OF THE INVENTION

As used herein, the following standard abbreviations are used throughout the specification to indicate specific amino acids:

| | |
|---|---|
| A = ala = alanine | R = arg = arginine |
| N = asn = asparagine | D = asp = aspartic acid |
| C = cys = cysteine | Q = gln = glutamine |
| E = glu = glutamic acid | G = gly = glycine |
| H = his = histidine | I = ile = isoleucine |
| L = leu = leucine | K = lys = lysine |
| M = met = methionine | F = phe = phenylalanine |
| P = pro = pro1ine | S = ser = serine |
| T = thr = threonine | W = trp = tryptophan |
| Y = tyr = tyrosine | V = val = valine |
| B = asx = asparagine or aspartic acid | |
| Z = glx = glutamine or glutamic acid | |

As used herein, the following standard abbreviations are used throughout the specification to indicate specific nucleotides: C=cytosine; A=adenosine; T=thymidine; G=guanosine; and U=uracil.

This invention provides a method for identifying a compound which inhibits HIV-1 entry into a cell:(a) determining the conformation of a crystal of the central cavity of HIV-1 gp120 in the presence of a ligand; (b) contacting the crystal of HIV-1 gp120 with the compound; (c) determining the conformation of the crystalized HIV-1 gp120 central cavity in the presence of the compound; and (d) comparing the conformation of the crystalized HIV-1 gp120 central cavity determined in step (c) with the conformation of the crystalized HIV-1 gp120 in step (a), so as to identify a compound which inhibits HIV-1 entry into the cell.

In one embodiment of the above method, the ligand in step (a) is soluble CD4 or a soluble CD4 mimetic.

This invention provides a compound identified by the above method.

In one embodiment of the methods described herein, the compound is not soluble CD4. In another embodiment, the compound is not soluble gp120. In a further embodiment, the compound is an antibody or portion of an antibody. In one embodiment, the antibody is a monocional antibody. In one embodiment, the antibody is a polyclonal antibody. In one embodiment, the antibody is a humanized antibody. In one embodiment, the antibody is a chimeric antibody. In one embodiment, the portion of the antibody comprises a light chain of the antibody. In one embodiment, the portion of the antibody comprises a heavy chain of the antibody. In one embodiment, the portion of the antibody comprises a Fab portion of the antibody. In one embodiment, the portion of the antibody comprises a F(ab′)₂ portion of the antibody. In one embodiment, the portion of the antibody comprises a Fd portion of the antibody. In one embodiment, the portion of the antibody comprises a Fv portion of the antibody. In one embodiment, the portion of the antibody comprises a variable domain of the antibody. In one embodiment, the portion of the antibody comprises one or more CDR domains of the antibody.

In one embodiment of the methods described herein, the compound is a polypeptide. In one embodiment, the compound is a peptide. In one embodiment, the compound is an oligopeptide.

In one embodiment of the methods described herein, the compound is nonpeptidyl agent. In one embodiment, nonpeptidyl agent is a carbohydrate. Such carbohydrate may be any carbohydrate known to one skilled in the art including but not limited to mannose, mannan or methyl-1-D-mannopyranoside. In one embodiment of the methods described herein, the compound is a small molecule or small molecular weight molecule. In one embodiment, the compound has a molecular weight less than 500 daltons.

This invention provides a composition which comprises the above compound and a carrier.

In one embodiment of the above composition, the carrier is a diluent, an aerosol, a topical carrier, an aqueous solution, a nonaqueous or a solid carrier. In another embodiment, the carrier is a pharmaceutically acceptable carrier.

As used herein, “carrier” includes but is not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like. The carriers include but are not limited to an aerosol, intravenous, oral or topical carrier. Carriers are well known to those skilled in the art.

This invention provides a compound which inhibits the cavity binding interaction between HIV-1 gp120 and CD4.

This invention provides a method of inhibiting HIV-1 infection of a cell comprising contacting the cell with an amount of the compound identified in the above method effective to inhibit HIV-1 infection, thereby inhibiting HIV-1 infection of the cell.

This invention provides a method of preventing HIV-1 infection in a subject comprising administering to the subject an amount of the compound identified in the above method effective to inhibit HIV-1 infection, thereby preventing HIV-1 infection in the subject.

This invention provides a method of treating HIV-1 infection in a subject comprising administering to the subject an amount of the compound identified in the above method effective to inhibit HIV-1 infection, thereby treating HIV-1 infection in the subject.

This invention provides a method of inhibiting HIV-1 infection of a cell comprising contacting the cell with an amount of the compound which inhibits the cavity binding interaction between HIV-1 gp120 and CD4 effective to inhibit HIV-1 infection, thereby inhibiting HIV-1 infection of the cell.

This invention provides a method of preventing HIV-1 infection in a subject comprising administering to the subject an amount of the compound which inhibits the cavity binding interaction between HIV-1 gp120 and CD4 effective to inhibit HIV-1 infection, thereby preventing HIV-1 infection in the subject.

This invention provides a method of treating HIV-1 infection in a subject comprising administering to the subject an amount of the compound which inhibits the cavity binding interaction between HIV-1 gp120 and CD4 effective to inhibit HIV-1 infection, thereby treating HIV-1 infection in the subject.

In one embodiment of the above methods, the subject is a mouse, rat, dog, guinea pig, ferret, rabbit, primate, and human. As used herein, “subject” means any animal or artificially modified animal capable of becoming infected with HIV-1 virus. Artificially modified animals include, but are not limited to, SCID mice with human immune systems. The subjects include but are not limited to mice, rats, dogs, guinea pigs, ferrets, rabbits, and primates. In the preferred embodiment, the subject is a human being.

The methods used for “soaking experiments” are well known to one of skill in the art (see for example, Current Biology (1994) 4: 784-797 and The EMBO Journal (1999) 18: 6249-6259).

As used herein, “effective amount” means an amount in sufficient quantities to either treat the subject or prevent the subject from becoming infected with HIV-1 virus. A person of ordinary skill in the art can perform simple titration experiments to determine what amount is required to treat the subject.

The subject invention has various applications which includes HIV-1 treatment such as treating a subject who has become afflicted with HIV-1. As used herein, “afflicted with the disease” means that the subject has at least one cell which has been infected by HIV-1. As used herein, “treating” means either slowing, stopping or reversing the progression of an HIV-1 disorder. In the preferred embodiment, “treating” means reversing the progression to the point of eliminating the disorder. As used herein, “treating” also means the reduction of the number of viral infections, reduction of the number of infectious viral particles, reduction of the number of virally infected cells, or the amelioration of symptoms associated with HIV-1.

Another application of the subject invention is to prevent a subject from contracting HIV-1. As used herein, “contracting HIV-1” means becoming infected with HIV-1, whose genetic information replicates in and/or incorporates into the host cells. Another application of the subject invention is to treat a subject who has become infected with HIV-1.

As used herein, “HIV-1 virus infection” means the introduction of KSHV genetic information into a target cell, such as by fusion of the target cell membrane with HIV-1 or an HIV-1 envelope glycoprotein cell. The target cell may be a bodily cell of a subject. In the preferred embodiment, the target cell is a bodily cell from a human subject.

This invention will be better understood from the Experimental Details that follow. However, one skilled in the art will readily appreciate that the specific methods and results discussed are merely illustrative of the invention as described more fully in the claims that follow thereafter.

Experimental Details

A. Materials and Methods

HXBc2: Data Collection and Refinement

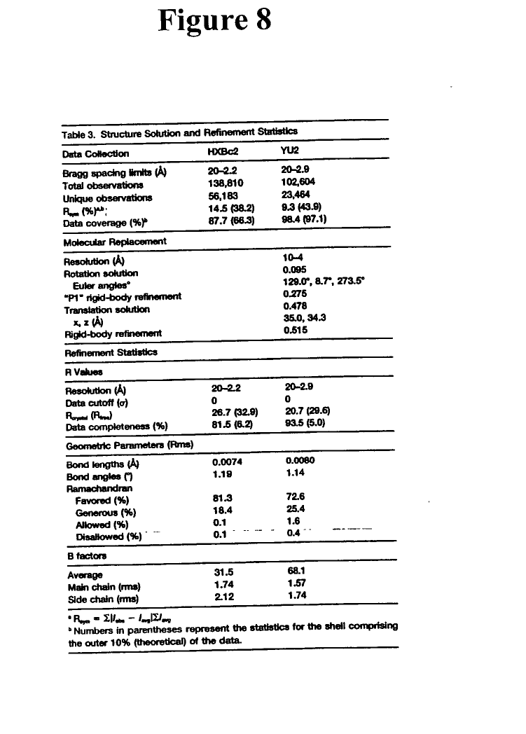

Crystals of the ternary complex of HXBc2 were grown, crosslinked, stabilized, and flash-cooled as previously described [Kwong, 1998; Kwong, 1999; Kwong, 1999]. The sole difference was an increase in the concentration of NaCl in the cryoprotectant stabilizer from 50 mM to 350 mM. Data were collected at beamline X4A, Brookhaven National Laboratory, using phosphoimaging plates and a Fuji BAS2000 scanner. Data processing and reduction were carried out with DENZO and SCALEPACK [Otwinowski, 1997]. The refined cell dimensions for the P222₁ crystals were a=71.247 Å, b=88.110 Å, and c=196.539 Å. Refinement was initiated with the Protein Data Bank coordinates, 1gc1.[Kwong, 1998] Torsional angle-simulated annealing, standard positional and individual isotropic B value refinement, and automatic water placement and deletion were carried out with CNS [Brunger, 1998]. Refinement details are given in FIG. 8. HXBc2 ternary complex coordinates have been deposited with the Protein Data Bank under accession code lG9M, and YU2 ternary complex coordinates have been deposited under accession code 1G9N.

YU2: Crystallization, Data Collection, Structural Determination and Refinement

The ternary complex of YU2 gp120 was prepared as described previously [Kwong, 1999], except that the YU2 core [Wu, 1996] was substituted for the HXBc2 core. Crystals were grown using the “hanging-droplet” method. The droplet was formed by combining 0.5 μl of protein solution (5 mg/ml ternary complex in 350 mM NaCl, 5 mM Tris pH 7.0) with 0-35 μl] of reservoir (250 μl of Hampton crystal screen reagent #18 combined with 50 μl of 0.5 M Na Acetate pH 4.5, 126 μl of 95% ethanol and 292 μl of water). An additional 45 μl of 5 M NaCl was subsequently added to the reservoir. Droplets were “streak-seeded” after two days by using a hair to transfer crushed microcrystals into the droplet. Visible crystals appeared within 3 days and grew to maximum size (150×50×50 μM) in 3-4 weeks. Crosslinking with vapor diffusion glutaraldehyde treatment [Lusty, 1998], stabilization (10% PEG 6,000, 10% isopropanol, 2.5% 2R,3R butandiol, 2.5% sucrose, 10% ethylene glycol, 100 mM Citrate/Hepes pH 5.7), and cryofreezing with paratone N (Exxon) were all carried out in a manner similar to that reported for the HXBc2 crystals [Kwong, 1999; Kwong, 1998]. Diffraction data were also collected at beamline X4A, but with a CCD detector. Data processing and reduction were carried out with DENZO and SCALEPACK [Otwinowski, 1997]. The refined cell dimensions for the C2 crystals were a=174.982 Å, b=81.706 Å, c=74.475 Å, and b=90.37°.

The refined 2.2 Å HXBc2 structure was used as a starting point for molecular replacement. Molecular replacement was carried out using 10-4 Å data. A rotational Patterson search (AMORE [Navaza, 1994]) produced a clear peak (9.5% correlation for the highest peak versus 8.9% for the next highest). Similar rotation solutions for either gp120-CD4 or the 17b Fab lent confidence to this solution. Translation searches with this initial rotational solution did not provide a clear solution. Taking advantage of the low symmetry, the rotational solution was rigid-body refined in a pseudo Pi lattice constructed by symmetry expansion of the C2 data. This pseudo Pi rigid-body refinement, carried out with XPLOR [Brunger, 1993], allowed CD4, gp120 and each of the four domains of the 17b antibody to move independently and raised the Patterson correlation to 27.5%. A translation search [Brunger, 1993] with this rigid-body refined model gave a Patterson correlation of 41.8%, which was raised to 51% upon further rigid body refinement. This model was subjected to torsional angle-simulated annealing, standard positional and individual isotropic B value refinement, and automatic water placement and deletion using the program CNS [Brunger, 1998]. Structure solution and refinement details are given in FIG. 8.

B. Results

Structure Solution and Refinement.

The HXBc2 ternary complex crystallized in the relatively rare spacegroup, P2221 [Wukovitz, 1995; Kwong, 1999]. Crystals generally diffracted to a Bragg limit of 3.5-2.5 Å, and we previously published the structure of this complex at 2.5 Å resolution. Despite extensive refinement, many details at this resolution remained ill-defined, including the identity of a large central density that dominated the Phe 43 cavity. In addition, several of the biologically important variable loops displayed high atomic mobility (B) factors, and were not accurately modeled.

By optimizing cryocooling techniques [Kwong, 1999], using a higher ionic strength stabilizing solution, and screening several dozen crystals, we were able to collect data to 2.2 Å resolution, a 150% increase in constraints over the previous 2.5 Å data. This data was highly anisotropic with an atomic mobility factor along the A axis of 16 Å² with respect to the C axis. Nonetheless, the electron density maps calculated with this data were of much better quality, enabling us to decipher more cryptic portions of the structure. The relatively high final R-factor of 26.8% (20-2.2 Å, all data>0, R-free=32.9%) reflected the anisotropy and relatively poor data quality. In contrast, the geometric parameters were well-satisfied and typified a high-quality 2.2 Å structure. The final model was composed of 8322 atoms comprising residues 93-397 and 410-492 of the HXBc2 core, residues 1-181 of CD4, and residues 1-214 of the light chain and 1-229 of the heavy chain of the 17b antibody; 14 N-acetylglucosamines and 2 fucose residues, 953 water molecules and an isopropanol were also modeled. The higher resolution data permitted more precise solvent modeling, which in turn created an overall more accurate low resolution model (at 4.5-5.0 Å, the R-factor was 17% with an R-free of 25%) enabling mobile portions of the complex to be accurately modeled. While the overall refined model was similar to the previously published structure, some of the more mobile portions differed significantly; the root-mean-square (rms) difference in backbone was 0.6 Å for atoms with B values less than 50 Å², and 2.5 Å for atoms with B values greater than 50 Å².

The YU2 ternary complex displayed unusual polymorphism. Six different crystals grew in the initial Hampton “Crystal Screen”, which increased to ten different morphologies after optimization. In conditions similar to those that ultimately yielded the 2.9 Å resolution C2 crystals, at least five other crystals grew: large single C2 plates that only diffracted to 6 Å, highly twinned needles that diffracted to 2.5 Å along the needle axis, but only to 6 Å otherwise, two needles in different P1 lattices that diffracted to 4 Å and 7 Å, and a blocky rectangular crystal that showed no diffraction at all. The combination of polymorphism, non-single crystals and generally weak diffraction frustrated the YU2 structure determination. Optimization was only possible by seeding to retain a particular crystal lattice.

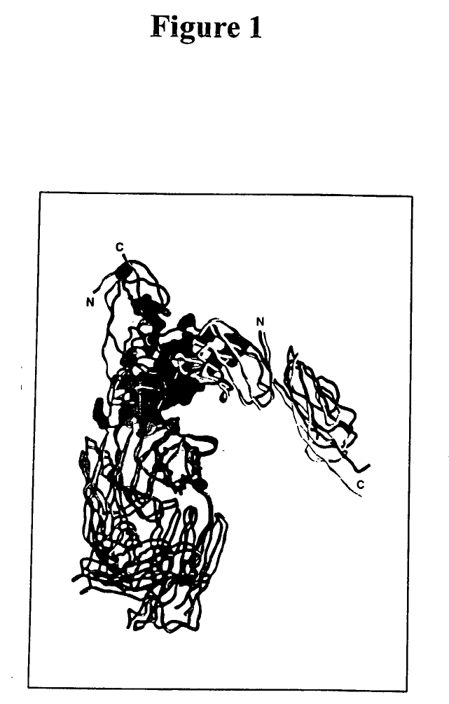

The YU2 ternary complex was solved using molecular replacement with the refined HXBc2 structure. It has been refined to a final R-value of 20.7% (20-2.9 Å data, data>0, R-free=29.6%). Data was also anisotropic, though this was less evident because the overall diffraction stopped at 2.9 Å. The final model, composed of 7719 atoms, comprises residues 86-492 of the YU2 gp120 core, residues 1-181 of CD4, and residues 1-214 of the light chain and 1-229 of the heavy chain of the 17b antibody, as well as 14 N-acetylglucosamines and 350 water molecules. A superposition of the HXBc2 and YU2 ternary complex structures is shown in FIG. 1.

Refined Structure of HXBc2 gp120.

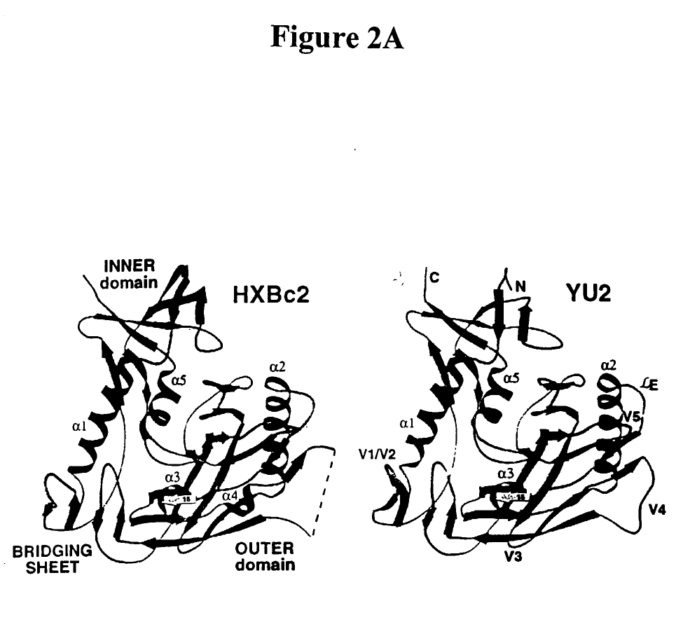

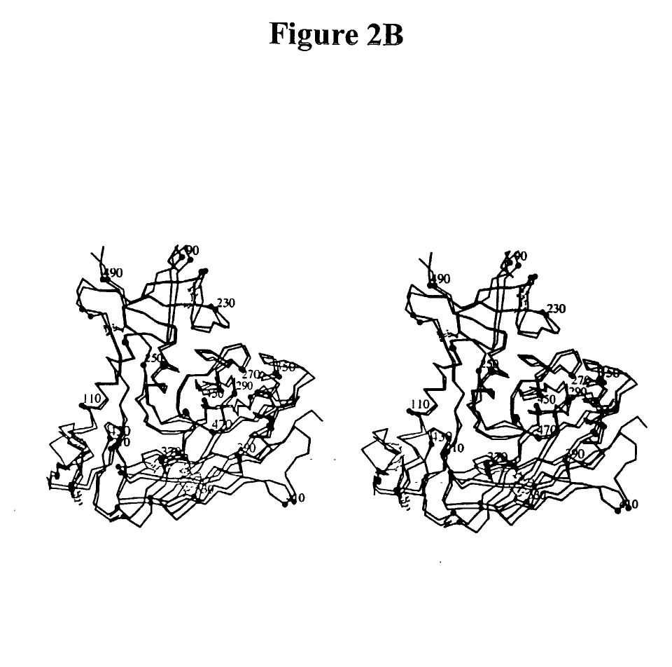

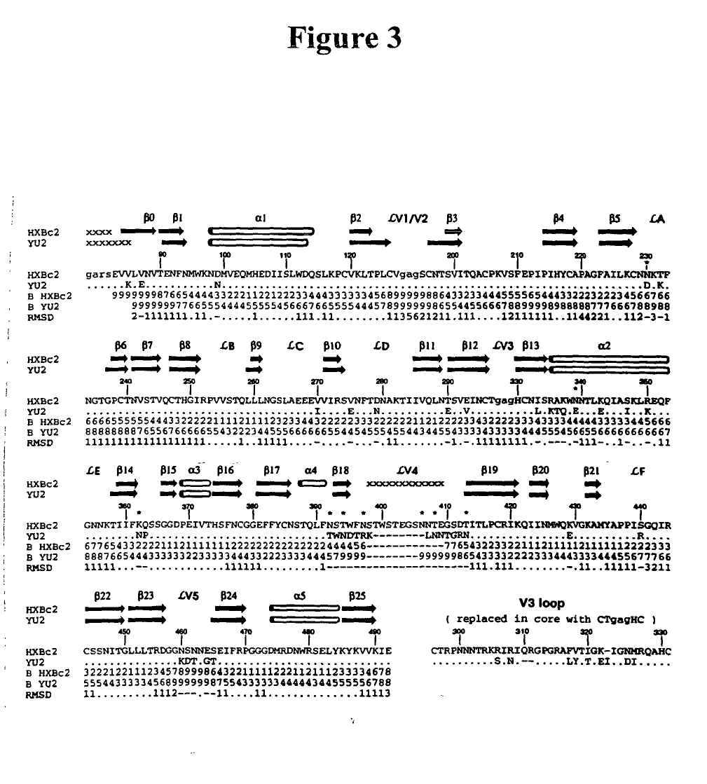

A ribbon diagram of the 2.2 Å refined HXBc2 core gp120 is shown in FIG. 2a, left panel. The structure resembles closely the previous published structure [Kwong, 1998]. All of the secondary structure is retained (FIG. 3). The only addition is a β-strand at the N terminus. This strand, which we term β0, is weakly ordered, as evidenced by high B factors. It hydrogen-bonds in an antiparallel manner with β7, completing a 4-on-2 antiparallel two-sheet subdomain.

Other than the N terminus (83-89), five other residues have been added to the structure—a single addition to the mostly disordered V4 loop (at 397), and four additional N-acetylglucosamines to asparagines at 88, 230, 241 and 463. Of the 18 potential sites of glycosylation in the HXBc2 core, 14 show sugar additions (although some of these may only be partially occupied), 3 are not seen (at 332, 356 and 397) and 1 falls in the disordered V4 loop. A large number of residues (51 out of 297) have shifted in the refinement more than 1.0 Å. Such shifts occur at the N terminus and loops (loop A and loop E, as well as the V1/V2 stem and V4 and V5 variable loops). Thus, the ordered core is essentially unchanged in the refinement, but some of the more mobile portions have now been accurately modeled.

Refined Structure of YU2 gp120.

A ribbon diagram of the 2.9 Å YU2 core gp120 is shown in FIG. 2a, right panel. The sequence of YU2 differs from HXBc2 in 41 positions of the core (FIG. 3). As may be expected from this 86% identity, Cα superposition shows that the two structures are virtually identical (FIG. 2b). Changes in the secondary structure of more than a single amino acid are found in only four places: the N terminus, where the tenuously ordered β0-strand in the HXBc2 structure appears to be displaced by a lattice contact with the N terminus of the 17b heavy chain; the V1/V2 stem, where the antiparallel hydrogen bonding is extended by three amino acids; the α4-helix, where the carbonyl of 388 makes a hydrogen-bond to the hydroxyl of Thr 392 (Asn in HXBc2) instead of to the 392 backbone nitrogen; and in the highly divergent V4 loop, which was disordered in the HXBc2 structure, but is fully ordered in YU2, along with its two sites of N-linked glycosylation.

In addition to the correlation in secondary structure, a strong correlation between B factors is also seen (FIG. 3), although the atomic mobilities of YU2 are generally quite a bit higher than in HXBc2, a consequence of the less ordered overall diffraction. Cα-rms deviations between YU2 and HXBc2 correlate with B factor, suggesting that differences between the two structures are in part related to their internal mobility.

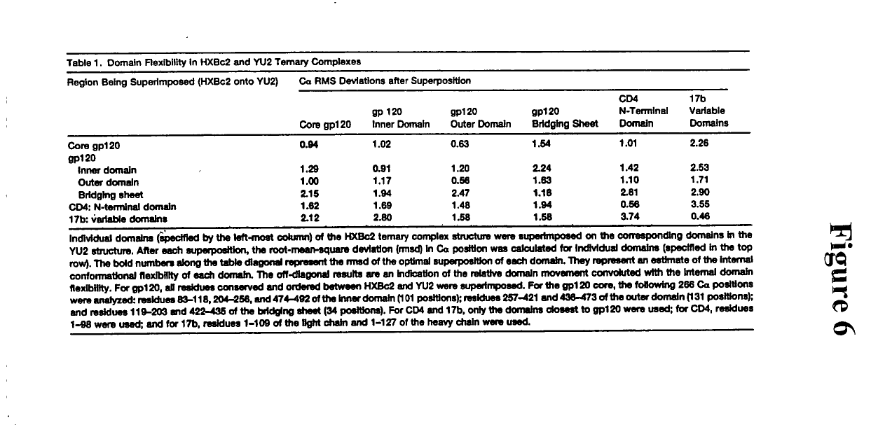

Sequence differences between YU2 and HXBc2 are primarily in the outer domain. of the 41 changes, 1 is in the bridging sheet (97% identity), 5 are in the inner domain (95% identity), and 35 are in the outer domain (79% identity). Surprisingly, overall domain structural deviations between HXBc2 and YU2 do not correlate with amino acid conservation. Superimposing the respective HXBc2 and YU2 domains shows that the outer domain has an rms deviation of roughly half that observed for the inner domain and the bridging sheet (FIG. 6). The deviations seen with the outer domain are of the same order as the deviations observed for the N-terminal domain of CD4 and the 17b variable domains. These results suggest that the deviations observed between HXBc2 and YU2 cores are not a result of sequence-related structural divergence, but rather a function of the intrinsic conformational mobility of the domains coupled with the different lattice environments of the two crystals.

For the bridging sheet, the flexibility in the V1/V2 stem in conjunction with a lattice contact with the second domain of CD4 results in a different orientation of the V1/V2 sterm and a high overall rms deviation. For the inner domain, the overall B factor is high; of the 101 amino acids in common between the HXBc2 and YU2 inner domain, only 26 have a Cα B factor of less than 50 Å². If superpositions are made with this ordered subfraction, the Cα rms deviation drops to 0.28 Å. In contrast, 70% of the outer domain amino acids have Ca B-factors of less than 50 Å².

Interestingly, the YU2 V3 loop, which is a primary determinant of both receptor usage and resistance to neutralization, is 72% identical (2 gaps) to the HXBc2 loop (counting from the conserved bridging cystines at the loop base). For the V4 loop, the conservation is 46% identity (1 gap) with alterations at 6 sites of N-linked glycosylation (FIG. 3). Despite these large differences in sequence, the V4 loop has not been shown to play an important role in determining the HXBc2 and YU2 neutralization-resistant phenotype.

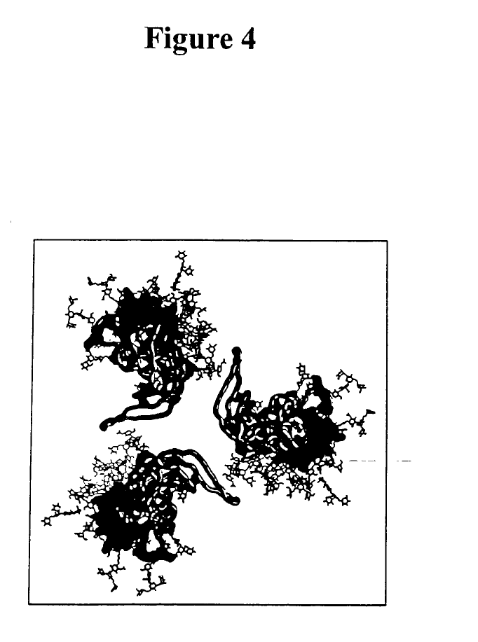

Given the overall high level sequence identity between these two HIV-1 isolates, the absolute magnitude of sequence differences does not appear to be critical for predicting functional phenotype or structural divergence. Nonetheless, the differences cluster on a surface that is predicted to be solvent exposed on the envelope oligomer (FIG. 4) [Kwong, 2000; Wyatt, 1998]. These changes will thus significantly alter the surface exposed to the immune system.

Receptor-gp120 Interactions.

Receptor-binding surfaces on the gp120 core are virtually identical in sequence between HXBc2 and YU2. There are two changes both in the CD4 binding region: at 279, Asp in HXBC2 changes to Asn; and at 460, where Asn changes to Lys. These two changes are in highly mobile regions and do not affect the binding of CD4 in any significant manner. Thus despite dramatic biochemical differences in 17b and CD4 binding to oligomeric HXBc2 and YU2, there are no significant sequence difference in the actual sites of binding on the core.

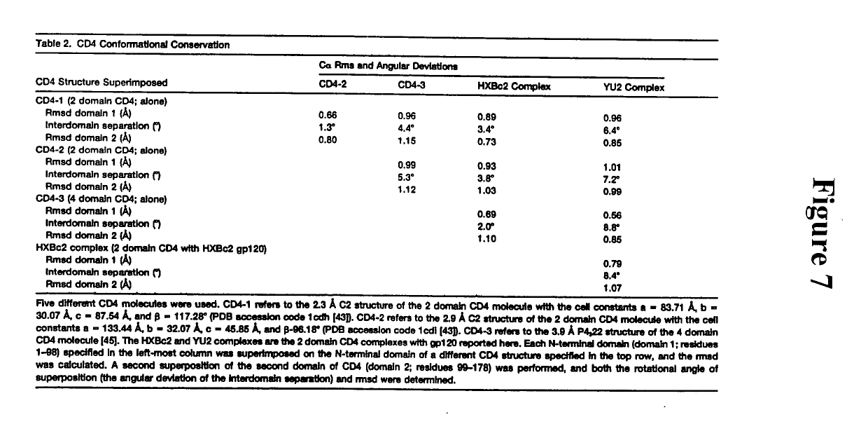

To detect structural variation in receptor binding, we analyzed domain superpositions, which illuminate not only internal domain rigidity, but also relative domain displacements. Visual inspection of the ternary complex after superposition of the gp120 core (FIG. 1), shows significant displacement of the second domain of CD4. Several CD4 structures have been determined: the two in complex with gp120 presented here, several of two-domain CD4 in two different lattices [Wang, 1990; Ryu, 1990 Ryu, 1994; Wu, 1996], and three of the entire extracellular portion (four domains) of CD4 [Wu, 1997 ]. Superpositions of representative structures showed that most were closely matched, indicating that the D1-D2 juncture is relatively rigid; for the three described in FIG. 7, the average angular displacement between domains was 3.4±1.4°. Surprisingly, in all superpositions, CD4 from the YU2 structure was an outlier, with an average angular deviation of 7.7±1.0°. This suggests that lattice forces in the YU2 crystal alter the relative disposition of the CD4 domains.

If lattice forces change the orientation of the relatively rigid CD4 domains, it would be reasonable to expect such forces to alter the orientation of gp120 with respect to CD4. But when core gp120 or either of its domains (inner or outer) are superimposed, the resultant position of CD4 is only 0.5-1.0 Å removed from optimal superposition (FIG. 6). These results suggest that the N-terminal domain of CD4 is rigidly held by gp120. The binding of CD4 to a recessed pocket may enforce its relative orientation.

For 17b, if the orientation of the N-terminal (variable) domains of 17b are analyzed after gp120 superposition, the displacement of 17b from its optimal superposition is roughly 2 Å, with an angular displacement of 6°. This displacement is considerably larger than that observed for CD4. The difference is apparent in FIG. 1, where the N-terminal domain of CD4 in the HXBc2 and YU2 structures superimposes well, but the 17b variable domains show considerable displacement. Because the 17b variable domains are internally rigid, with an rmsd between HXBc2 and YU2 of only 0.46 Å, their displacement with respect to gp120 suggests flexibility in the 17b binding surface on gp120. This surface, which overlaps the chemokine receptor binding site [Rizzuto, 1998], is relatively flat, and thus may not impart the same orientational stringency as the recessed CD4 binding site. This flatness, coupled with the flexible nature of the bridging sheet itself, suggests orientational flexibility in the chemokine receptor-binding site.

The Central Cavity Between CD4 and gp120.

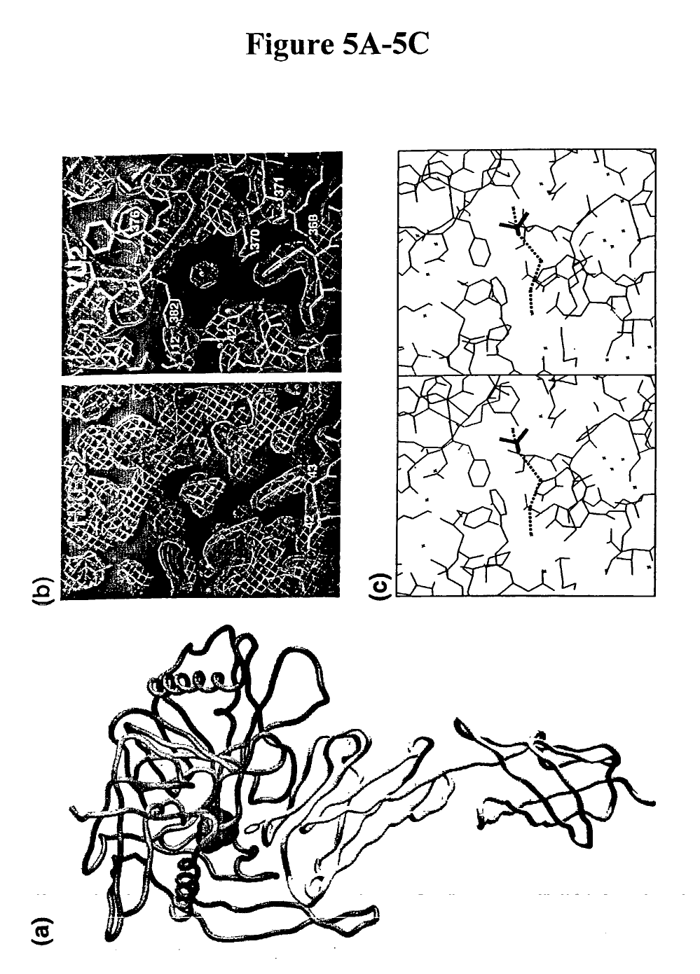

When the 2.5 Å ternary complex was reported [Kwong, 1998], a central mystery was the identity of the density dominating the Phe 43 cavity, at the nexus of the interaction between CD4, the inner domain, the outer domain and the bridging sheet. Analysis of the solvent-accessible surface of the YU2 complex revealed an interfacial cavity, at the same position as in HXBc2 (FIG. 5a). An inspection of this cavity showed that it too contained a central density (FIG. 5b). Residues which bound this cavity were all conserved between HXBc2 and YU2.

Refinement of the HXBc2 structure with 2.2 Å data, coupled with automatic water placement, modeled the Phe 43 cavity density as 3 water molecules, displaced 2. 3, 2.4 and 2.8 Åfrom each other. The water with closest distances to the others, displayed a B factor that was less than half of the others, and showed hydrogen bonding to two other well resolved waters in the cavity. This 3-water structure models extremely well an isopropanol (with the exception of a central carbon), a major component of the crystallization medium. In isopropanol, the hydroxyl-peripheral carbon distance (2.4 Å) is shorter than the carbon-carbon distance (2.5 Å); the hydroxyl has more electrons and would appear to have a lower B factor if modeled as an atom equivalent to the carbons; only the hydroxyl should show hydrogen bonding. To test this hypothesis, two isopropanol molecules were place into the central density, with the central carbon placed in opposite orientations, and the overall structures refined. In one orientation, both the R-factor and the free-R was lower than the 3-water model; in the other, both were higher. The low R-factor orientation is shown in FIG. 5b. The central carbon makes a van der Waals contact with the sidechain of Val 255. (Interestingly, refinement of the two different isopropanol orientations with 2.2 Å data truncated at 2.5 Å, showed no discrimination in overall R-values. This suggests that the extension of the refinement to 2.2 Å resolution was crucial for isopropanol identification.)

The refined B factors of the isopropanol were comparable to the surrounding protein sidechains. This suggests that it is not only well ordered, but of high occupancy. Because the gp120 core/D1D2 CD4/17b complex was formed prior to crystallization [Kwong, 1999], the isopropanol had to penetrate into the cavity from the exterior solvent. Ordered water molecules, demarking a path out of the cavity between the bridging sheet and the inner domain, suggest a route of entry (FIG. 5c). The observed flexibility of both the bridging sheet and this portion of the inner domain support this notion.

C. Discussion

Primary and Laboratory-Adapted Viruses.

The ability of HIV-1 to evade immune clearance is a hallmark of the virus as well as the basis of its ability to maintain a persistent, ultimately fatal, infection. Evasion of the humoral immune response is due to the structure of the HIV-1 envelope glycoproteins, particularly the exterior gp120 envelope glycoprotein.

The gp120 glycoprotein also functions in virus entry. In order to bind receptor, it must expose constant regions; but in order to avoid immune detection, it must hide constant regions. This functional dilemma results in viruses that are optimized differently depending on the selective conditions under which the virus is grown. In an individual with a vigilant immune response, evasion of this response is essential. Primary, clinical HIV-1 isolates display high resistance to neutralization, relative to viruses extensively passaged in culture. Primary isolates differ in sensitivity to neutralization, probably dependent upon the history of virus passage and upon the antibody repertoire elicited in the host. For example simian-human immunodeficiency virus (SHIV) chimera, which contain HIV-1 envelope glycoproteins, become very resistant to neutralization by antibodies after being passaged in monkeys [Cheng-Mayer, 1999; Etemad-Moghadam, 1999]. YU2 represents one of the more difficult primary isolates to neutralize, possibly because it has never been passaged in culture.

Our initial analysis of the structure of core gp120 revealed multiple potential mechanisms of immune evasion [Kwong, 1998; Wyatt, 1998]. These included loop variation, carbohydrate cloaking, conformational change, islands of variations and steric occlusion, both by oligomerization and mobile loop interference. Analysis of the structures of the YU2 core and the laboratory-adapted HXBc2 core shows that both display all of the potential mechanisms of immune evasion. How then to explain the dramatic difference in sensitivity to neutralization between YU2 and HXBc2 HIV-1?

The determinants of the changes between primary and laboratory-adapted viruses have been examined for several different virus pairs [Etemad-Moghadam, 1999; Cheng-Mayer, 1999]. In almost all cases, the V1/V2 and V3 loops cooperate to determine most of the resistant phenotype. Differences in antibody binding to envelope glycoproteins show up only on oligomers, not monomers [Parren, 1998; Sullivan, 1995; Fouts, 1997].

This suggests that while mechanisms of immune evasion are present on the monomeric core, they are operational in the context of the assembled envelope trimer. With HXBc2 and YU2, the substitution of the V3 loop of YU2 into HXBc2, a change at only 11 amino acids, results in a virus that is neutralization resistant [Sullivan, 1998]. Apparently, subtle differences in a specific part of the gp120 monomer can result in significant effects on antibody binding to the assembled trimer. Interactions among the major variable loops of each subunit are likely to play a role in masking the conserved gp120 core epitopes.

The difference in neutralization resistance between primary and laboratory-adapted isolates is exemplified by the difference in binding of the 17b antibody, which is present in both of the ternary complexes described here. 17b binds well to monomeric core and monomeric full-length gp120 from both YU2 and HXBc2. It also binds oligomeric HXBc2 envelope glycoproteins and neutralizes HXBc2 virus. However, 17b binding to oligomeric YU2 envelope glycoproteins or YU2 virus is less efficient, and V3 loop substitution changes the phenotype of 17b neutralization of HXBc2 into that of YU2.

In terms of CD4 binding, numerous reports note an increase in the ability to bind CD4 (or to be neutralized by sCD4) or to infect cells with low levels of CD4 expression as a consequence of laboratory adaptation [Zhang, 1997; Kabat, 1994; Platt, 2000; Kozak, 1997; Bannert, 2000]. Measurements of the affinity for CD4 of primary and laboratory-adapted isolates, however, shows that differences exist only in the context of the oligomer [Brighty, 1991]. Analysis of the thermodynamics of CD4 binding to full-length YU2 and laboratory-adapted gp120 shows that both exhibit the same unusual thermodynamics ([Myszka, 2000] and M. Doyle personal communication). Structural comparison of CD4 binding to YU2 and to HXBc2 shows that the orientation of CD4 is rigidly determined by gp120, and is the same for both primary and laboratory-adapted gp120 cores.

These results suggest that the determinants which encode for differences in neutralization resistance and CD4 binding affinity do not reside on the core but in oligomeric contacts and/or on loops which emanate from the core. These contacts and/or loops serve to control or to modulate the action of the core. Structural analysis of the YU2 and HXBc2 cores shows that most of the structural differences are a result of inherent flexibility coupled to different lattice contacts. In the same manner that lattice contacts alter the core, oligomeric contacts and/or loops may use the structural flexibility inherent in gp120 to modulate the properties of the core. Such modulation would permit rapid adaption since the underlying mechanisms of immune evasion, receptor binding and virus entry would remain intact.

Generality of gp120 Structure.

The determination of a second gp120 ternary complex permits analysis of the generality of many of the specific structural features observed in the inital HXBc2 structure. The conservation of secondary structure, receptor binding features and the central Phe 43 cavity is presented above; other biologically important features are addressed below.

Cavities.

Because cavities are calculated as the difference of solvent accessible surfaces, they are sensitive to small positional deviations. We find that the small cavities internal to the gp120 structure, especially at the interface between the inner and outer domain, are not conserved, i.e. are subject to considerable variation. The one internal gp120 cavity, that is conserved is at the interface between the inner and outer domains, about 10 A proximal to the core termini from the Phe-43 cavity. The large shallow interfacial cavity observed in the HXBc2 structure between CD4 and gp120, is not a cavity in the YU2 structure, with side chain conformational differences altering its shape. These differences, however, should not affect the function of this outer “island of variation” in immune evasion [Bewley, 1999].

Atomic Mobility.

Some correlation is observed in the atomic mobilities of the HXBc2 and YU2 structures, although this may be influenced by lattice contacts. The N/C terminal region of YU2 exhibits much higher relative B factors than the equivalent region in HXBc2. The lattice packing disruption of the β0-strand may account for some of this. For the carbohydrate, virtually all of the N-acetylglucosamines at sites of N-linked glycosylations are ordered in both HXBc2 (14 out of 18) and YU2 (14 out of 18). This percentage is higher if unoccupied sites or sites where mainchain atoms are disordered are excluded. These results suggest that many of the peptide-proximal gp120 carbohydrates are structurally integral to the surface of the core.

Orientation of Loops.

Some of the positions of the loops (such as the V5 loop) are maintained even though the sequence is divergent. Other loops (such as the terminus of the V1/V2 stem) assume different conformations even though the sequence is conserved. Still others, like the V4 loop are divergent in both sequence and structure. In general it appears that the loops are mobile and conservation is either accidental or the result of similar lattice packing (the V5 loop, for example, makes a lattice contact with the constant portion of a symmetry-related 17b heavy chain that is conserved in both crystal lattices).

Phe 43 Cavity: Functionality and Therapeutic Target.

As discussed above, the Phe 43 cavity is conserved in both of the HXBc2 and YU2 ternary complexes. Similarly positioned hydrophobic interfacial cavities are observed in the structures of poliovirus and its cellular receptor, PVR [He, 2000; Belnap, 2000], and rhinovirus and its receptor ICAM-1 [Kolatkar, 1999]. In these latter cases, the cavities are substantially larger and prior to receptor binding appear to be filled by a “pocket factor”, which is thought to be a lipid molecule. (See e.g. Grant R. A., Curr. Biol 1994 September1;4(9):784-97 for drug design implications of structures of poliovirus complexes and anti-Oviral drugs and Joseph-McCarthy D. et al., Proteins 29:32-58 (1997).) These pocket factors appear to be expelled upon receptor binding, creating an empty hydrophobic cavity. The collapse of this cavity is thought to drive conformational changes associated with virus entry.

With gp120, there is no evidence for the existence of a “pocket” in the absence of CD4; rather the unusual thermodynamics of CD4 binding suggest that the cavity may only be formed when CD4 is bound [Myszka, 2000]. The functional importance of the HIV-1 Phe 43 cavity remains to be determined. In SIV, Ser 375 is replaced with Trp, a change that would be expected to fill the cavity; differences between HIV-1 and SIV in terms of requirements for CD4 usage might be related to these structural differences. Whatever its function, sequence analysis suggests that the Phe 43 cavity is conserved in HIV-1. Its presence in both HXBc2 and YU2 along with similarly positioned cavity densities argues for functional relevance.

The presence of isopropanol in the HXBc2 crystals suggests that lead compounds directed to this central cavity can be analyzed by simple soaking experiments. Cavity binding compounds could be used to investigate the function of this conserved feature. Lead compounds could be attached to sCD4 (or a CD4-mimetic) to enhance antagonist binding. Finally, if cavity binding compounds stabilize the CD4-bound conformation of gp120 in the absence of CD4, they may aid in the presentation of the conformationally disguised, functionally conserved, CD4 binding surface to the immune system.

Biological Implications

The structure of the HIV gp120 envelope glycoprotein visualizes two overlapping sets of machinery. One, involved in entry, binds to both CD4 and a chemokine receptor and transmits a signal to gp41 to initiate membrane fusion. The other uses loop variation, steric occlusion, conformational change and a carbohydrate cloak to evade the immune system. Analysis of the 2.9 Å structure of the CCR5-using primary YU2 isolate and the 2.2 Å structure of the CXCR4-using laboratory-adapted HXBc2 isolate suggests that both sets of machinery are present in both viruses. CD4 binding is conserved as are the presence of topological mismatches between interacting surfaces. The chemokine receptor binding surface shows variance, which we attribute to intrinsic flexibility and crystal lattice contacts, not to sequence-related structural changes. Mechanisms of immune evasion appear to be operational as well. Our structural results coupled to chimeric substitution and mutational analysis suggest that determinants which encode for neutralization resistance, chemokine receptor usage and CD4 binding affinity, do not reside on the core but in the major variable loops that emanate from the core. Such peripheral control in sequence-malleable regions allows the complex underlying core machinery to remain intact, and thus provides greater opportunities for viral adaptation to selective pressures.

The solution of two different gp120 core structures permits an analysis of the generality of specific features. Differences are observed in the orientations of loops and in atomic mobilities, but unusual characteristics such as a precisely oriented CD4, ordered N-linked carbohydrate, and a hydrophobic interfacial cavity are conserved. This cavity resembles cavities observed in the structures of poliovirus-receptor and rhinovirus-receptor complexes (see Grant, R. A. et al and Joseph-McCarthy, D. et al. Proteins 1997 September; 29(1):32-58 for use of multiple cupy simultaneous search (MCSS) method to design a new class of picornavirus capsid binding drugs) Therapeutic cavity-binding inhibitors have been identified for both polio and rhinovirus; the presence of a well-ordered isopropanol in the hydrophobic gp120-CD4 cavity suggests that such an approach may work with HIV-1. (see Structures of poliovirus complexes with anti-viral drugs: implications for viral stability and drug design. Grant R. A. et al. Curr. Biol. 1994 September. 1;4(9):784-797))

### REFERENCES

8. Barre-Sinoussi, F., Chermann, J. C., Rey, F., Nugeyre, M. T., Chamaret, S., Gruest, J., Dauguet, C., Axler-Blin, C., Vezinet-Brun, F., Rouzioux, C., Rozenbaum, W. & Montagnier, L. (1983). Isolation of a T-lymphotropic retrovirus from a patient at risk for acquired immunodeficiency syndrome (AIDS). Science 220, 868-871.

2. Gallo, R. C., Salahuddin, S. Z., Popovic, M., Shearer, G. M., Kaplan, M., Haynes, B. F., Palker, T. J., Redfield, R., Oleske, J., Safai, B., White, G., Foster, P. & Markham, P. D. (1984). Frequent detection and isolation of cytopathic retroviruses (HTLV-III) from patients with AIDS and at risk for AIDS. Science 224, 500-503.

3. Veronese, F. D., DeVico, A. L., Copeland, T. D., Oroszlan, S., Gallo, R. C. & Sarngadharan, M. G. (1985). Characterization of gp41 as the transmembrane protein coded by the HTLV-III/LAV envelope gene. Science 229, 1402-1405.

4. Robey, W. G., Safai, B., Oroszlan, S., Arthur, L. O., Gonda, M. A., Gallo, R. C. & Fischinger, P. J. (1985). Characterization of envelope and core structural gene products of HTLV-III with sera from AIDS patients. Science 228, 593-595.

5. Allan, J. S., Coligan, J. E., Barin, F., McLane, M. F., Sodroski, J., Rosen, C. A., Haseltine, W. A., Lee, T. H. & Essex, M. (1985). Major glycoprotein antigens that induce antibodies in AIDS patients are encoded by HTLV-III. Science 228, 1091-1094.

6. Dalgleish, A. G., Beverley, P. C., Clapham, P. R., Crawford, D. H., Greaves, M. P. & Weiss R. A. (1984) The CD4 (T4) antigen is an essential component of the receptor for the AIDS retrovirus. Nature 312, 763-767.

7. Feng, F., Broder, C. C., Kennedy, P. E. & Berger, E. A. (1996). HIV-1 entry co-factor: functional cDNA cloning of a seven-transmembrane, G protein-coupled receptor. Science 272, 872-877.

8. Deng, H., Liu, R., Ellmeier, W., Choe, S., Unutmaz, D., Burkhart, M., Di Marzio, P., Marmon, S., Sutton, R. E., Hill, C. M., Davis, C. B., Peiper, S. C., Schall, T. J., Littman, D. R. & Landau, N. R. (1996). Identification of a major co-receptor for primary isolates of HIV-1. Nature 381, 661-666.

9. Choe, H., Farzan, M., Sun, Y., Sullivan, N., Rollins, B., Ponath, P. D., Wu, L., Mackay, C. R., LaRosa, G., Newman, W., Gerard, N., Gerard, C. & Sodroski, J. (1996). The beta-chemokine receptors CCR3 and CCR5 facilitate infection by primary HIV-1 isolates. Cell 85, 1135-1148.

10. Dragic, T., Litwin, V., Allaway, G. P., Martin, S. R., Huang, Y., Nagashima, K. A., Cayanan, C., Maddon, P. J., Koup, R. A., Moore, J. P. & Paxton, W. A. (1996). HIV-1 entry into CD4+cells is mediatd by the chemokine receptor CC-CKR-5. Nature 381, 667-673.

11. Alkhatib, G., Combadiere, C., Broder, C. C., Feng, Y., Kennedy, P. E., Murphy, P. M. & Berger, E. A. (1996). CC CKR5: a RANTES, MIP-1alpha, MIP-1beta receptor as a fusion cofactor for macrophage-tropic HIV-1. Science 272, 1955-1958.

12. Doranz, B. J., Rucker, J., Yi, Y., Smyth, R. J., Samson, M., Peiper, S. C., Parmentier, M., Collman, R. G. & Doms, R. W. (1996). A dual-tropic primary HIV-1 isolate that uses fusin and the beta-chemokine receptors CKR-5, CKR-3, and CKR-2b as fusion cofactors. Cell 85, 1149-1158.

13. Chan, D. C., Fass, D., Berger, J. M. & Kim, P. S. (1997). Core structure of gp41 from the HIV envelope glycoprotein. Cell 89, 263-273.

14. Weissenhorn, W., Dessen, A., Harrison, S. C., Skehel, J. J. & Wiley, D. C. (1997). Atomic structure of the ectodomain from HIV-1 gp41. Nature 387, 426-430.

15. Kwong, P. D., Wyatt, R., Robinson, J., Sweet, R. W., Sodroski, J. & Hendrickson, W. A. (1998). Structure of an HIV gp120 envelope glycoprotein in complex with the CD4 receptor and a neutralizing human antibody. Nature 393, 648-659.

16. Myszka, D. G., Sweet, R. W., Hensley, P., Brigham-Burke, M., Kwong, P. D., Hendrickson, W. A., Wyatt, R., Sodroski, J. & Doyle, M. L. (2000). Energetics of the HIV gp120-CD4 binding reaction. Proc. Natl. Acad. Sci. USA 97, 9026-9031.

17. Wyatt, R., Kwong, P. D., Desjardins, E., Sweet, R. W., Robinson, J., Hendrickson, W. A. & Sodroski, J. (1998). The antigenic structure of the human immunodeficiency virus gp120 envelope glycoprotein. Nature 393, 705-711.

18. Wyatt, R. & Sodroski, J. (1998). The HIV-1 envelope glycoproteins: fusogens, antigens and immunogens. Science 280, 1884-1888.

19. Bewley, M. C., Springer, K., Zhang, Y. B., Freimuth, P. & Flanagan, J. M. (1999). Structural analysis of the mechanism of Adenovirus binding to its human cellular receptor, CAR. Science 286, 1579-1583.

20. Cocchi, R., DeVico, A. L., Garzino-Demo, A., Cara, A., Gallo, R. C. & Lusso, P. (1996). The V3 domain of the HIV-1 gp120 envelope glycoprotein is critical for chemokine-mediated blockade of infection. Nat. Med. 2, 1244-1247.

21. Bieniasz, P. D., Fridell, R. A., Aramori, I., Ferguson, S. S., Caron, M. G. & Cullen, B. R. (1997). HIV-1-induced cell fusion is mediated by multiple regions within both the viral envelope and the CCR-5 co-receptor. EMBO J. 16, 2599-2609.

22. Speck, R. F., Wehrly, K., Platt, E. J., Atchison, R. E., Charo, I. F., Kabat, D., Chesebro, B. & Goldsmith, M. A. (1997). Selective employment of chemokine receptors as human immunodeficiency virus type 1 coreceptors determined by individual amino acids within the envelope V3 loop. J. Virol. 71, 7136-7139.

23. Rizzuto, C. D., Wyatt, R., Hernandez-Ramos, N., Sun, Y., Kwong, P. D., Hendrickson, W. A. & Sodroski, J. (1998). A conserved human immunodeficiency virus gp120 glycoprotein structure involved in chemokine receptor binding. Science 280, 1949-1953.

24. Farzan, M., Mirzabekov, T., Kolchinsky, P., Wyatt, R., Cayabyab, M., Gerard, N. P., Gerard, C., Sodroski, J. & Choe, H. (1999). Tyrosine sulfation of the amino ternimus of CCR5 facilitates HIV-1 entry. Cell 96, 667-676.

25. Wrin, T., Loh, T. P., Vennari, J. C., Schuitemaker, H. & Nunberg, J. H. (1995). Adaptation to persistent growth in the H9 cell line renders a primary isolate of human immunodeficiency virus type 1 sensitive to neutralization by vaccine sera. J. Virol. 69, 39-48.

26. Sullivan, N., Sun, Y., Li, J., Hofmann, W. & Sodroski, J. (1995). Replicative function and neutralization sensitivity of envelope glycoproteins from primary and T-cell line-passaged human immunodeficiency virus type 1 isolates. J. Virol. 69, 4413-4422.

27. Koito, A., Harrowe, G., Levy, J. A. & Cheng-Mayer, C. (1994). Functional role of the V1/V2 region of human immunodeficiency virus type 1 envelope glycoprotein gp120 in infection of primary macrophages and soluble CD4 neutralization. J. Virol. 68, 2253-2259.

28. Hwang, S. S., Boyle, T. J., Lyerly, H. K. & Cullen, B. R. (1992). Identification of envelope V3 loop as the major determinant of CD4 neutralization sensitivity of HIV-1. Science 257, 535-537.

29. Morikita, T., Maeda, Y., Fujii, S., Matsushita, S., Obaru, K. & Takatsuki, K. (1997). The V1/V2 region of human immunodeficiency virus type 1 modulates the sensitivity to neutralization by soluble CD4 and cellular tropism. AIDS Res. Hum. Retroviruses 13, 1291-1299.

30. Sullivan, N., Sun, Y., Binley, J., Lee, J., Barbas, C. F., Parren, P. W., Burton, D. R. & Sodroski, J. (1998). Determinants of HIV-1 envelope glycoprotein activation by sCD4 and monoclonal antibodies. J. Virol. 72, 6332-6338.

31. Schonning, K., Jansson, B., Olofsson, S. & Hansen, J. E. (1996). Rapid selection for an N-linked oligosaccharide by monoclonal antibodies directed against the V3 loop of human immunodeficiency virus type 1. J. Gen. Virol. 77, 753-758.

32. Ly, A. & Stamatatos, L. (2000). V2 loop glycosylation of the human immunodeficiecy virus type 1 SF162 envelope facilitates interaction of this protein with CD4 and CCR5 receptors and protects the virus from neutralization by anti-V3 loop and anti-CD4 binding site antibodies. J. Virol. 74, 6769-6776.

33. Fouts, T. R., Binley, J. M., Trkola, A., Robinson, J. E. & Moore, J. P. (1997). Neutralization of the human immunodeficiency virus type 1 primary isolate JR-FL by human monoclonal antibodies correlates with antibody binding to the oligomeric form of the envelope glycoprotein complex. J. Virol. 71, 2779-2785.

34. Parren, P. W., Moore, J. P., Burton, D. R. & Sattentua, Q. J. (1999). The neutralizing antibody response to HIV-1: viral evasion and escape from humoral immunity. AIDS Suppl 13A, S137-62.

35. Li, Y., Kappes, J. C., Conway, J. A., Price, R. W., Shaw, G. M. & Hahn, B. H. (1991). Molecular characterization of human immunodeficiency virus type 1 cloned directly from uncultured human brain tissue: identification of replication-competent and defective viral genomes. J. Virol. 65, 3973-3985.

36. Wukovitz, S. W. & Yeates, T. O. (1995). Why protein crystals favour some space-groups over others. Nature Struct. Biol. 2, 1062-1067.

37. Kwong, P. D., Wyatt, R., Desjardins, E., Robinson, J., Culp, J. S., Hellmig, B. D., Sweet, R. W., Sodroski, J. & Hendrickson, W. A. (1999). Probability analysis of variational crystallization and its application to gp120, the exterior envelope glycoprotein of type 1 human immunodeficiency virus (HIV-1). J. Biol. Chem. 274, 4115-4123.

38. Kwong, P. D. & Liu, Y. (1999). Use of cryoprotectants in combination with immiscible oils for flash cooling macromolecular crystals. J. Appl. Cryst. 32, 102-105.

39. Kwong, P. D., Wyatt, R., Sattentau, Q. J., Sodroski, J. & Hendrickson, W. A. (2000). Oligomeric modeling and electrostatic analysis of the gp120 envelope glycoprotein of human immunodeficiency virus (HIV). J. Virol. 74, 1961-1972.

40. Stewart, J. J., Watts, P. & Litwin, S. (1999). Profile of a quasispecies: The selected mutational landscape of HIV and Influenza. In Theory and Mathematics in Biology and Medicine:487 Vrije Universiteit, Amsterdam.

41. Wang, J. H., Yan, Y. W., Garrett, T. P., Liu, J. H., Rodgers, D. W., Garlick, R. L., Tarr, G. E., Husain, Y., Reinhertz, E. L. & Harrison, S. C. (1990). Atomic structure of a fragment of human CD4 containing two immunoglobulin-like domains. Nature 348, 411-418.

42. Ryu, S. E., Kwong, P. D., Truneh, A., Porter, T. G., Arthos, J., Rosenberg, M., Dai, X. P., Xuong, N. H., Axel, R., Sweet, R. W. & Hendrickson, W. A. (1990). Crystal structure of an HIV-binding recombinant fragment of human CD4. Nature 348, 419-426.

43. Ryu, S. E., Truneh, A., Sweet, R. W. & Hendrickson, W. A. (1994). Structures of an HIV and MHC binding fragment from human CD4 as refined in two crystal lattices. Structure 2, 59-74.

44. Wu, H., Myszka, D. G., Tendian, S. W., Brouillette, C. G., Sweet, R. W., Chaiken, I. M. & Hendrickson, W. A. (1996). Kinetic and structural analysis of mutant CD4 receptors that are defective in HIV gp120 binding. Proc. Natl. Acad. Sci. USA 93, 15030-15035.

45. Wu, H., Kwong, P. D. & Hendrickson, W. A. (1997). Dimeric association and segmental variability in the structure of human CD4. Nature 387, 527-530.

46. Cheng-Mayer, C., Brown, A., Harouse, J., Luciw, P. A. & Mayer, A. J. (1999). Selection for neutralization resistance of the simian/human immunodeficiency virus SHIVSF33A variant in vivo by virtue of sequence changes in the extracellular envelope glycoprotein that modify N-linked glycosylation. J. Virol. 73, 5294-5300.

47. Etemad-Moghadam, B., Sun, Y., Nicholson, E. K., Karlsson, G. B., Schenten, D. & Sodroski, J. (1999). Determinants of neutralization resistance in the envelope glycoproteins of a simian-human immunodeficiency virus passaged in vivo. J. Virol. 73, 8873-8879.

48. Parren, P. W., Mondor, I., Naniche, D., Ditzel, H. J., Klasse, P. J., Burton, D. R. & Sattentau, Q. J. (1998). Neutralization of human immunodeficiency virus type 1 by antibody to gp120 is determined primarily by occupancy of sites on the virion irrespective of epitope specificity. J. Virol. 72, 3512-3519.

49. Moore, J. P., McKeating, J. A., Huang, Y. X., Ashkenazi, A. & Ho, D. D. (1992). Virions of primary human immunodeficiency virus type 1 isolates resistant to soluble CD4 (sCD4) neutralization differ in sCD4 binding and glycoprotein 120 retention from sCD4-sensitive isolates. J. Virol. 66, 235-243.

50. Kabat, D., Kozak, S. L., Wehrly, K. & Chesebro, B. (1994). Differences in CD4 dependence for infectivity of laboratory-adapted and primary patient isolates of human immunodeficiency virus type 1. J. Virol. 68, 2570-2577.

51. Platt, E. J., Kozak, S. L. & Kabat, D. (2000). Critical role of enhanced CD4 affinity in laboratory adaptation of human immunodeficiency virus type 1. AIDS Res. Hum. Retroviruses 16, 871-882.

52. Kozak, S. L., Platt, E. J., Madani, N., Ferro, F. E., Peden, K. & Kabat, D. (1997). CD4, CXCR-4 and CCR-5 dependencies for infections by primary patient and laboratory-adapted isolates of human immunodeficiency virus type 1. J. Virol. 71, 873-882.

53. Bannert, J., Schenten, D., Craig, S. & Sodroski, J. (2000). The level of CD4 expression limits infection of primary Rhesus monkey macrophages by a T-tropic simian immunodeficiency virus and macrophagetropic human immunodeficiency viruses. J. Virol. 74, in press.

54. Brighty, D. W., Rosenberg, M., Chen, I. S. & Ivey-Hoyle, M. (1991). Envelope proteins from clinical isolates of human immunodeficiency virus type 1 that are refractory to neutralization by soluble CD4 possess high affinity for the CD4 receptor. Proc. Natl. Acad. Sci. USA 88, 7802-7805.

55. Perutz, M. F. (1979). Regulation of oxygen affinity of hemoglobin, influence of structure of the globin on the heme iron. Ann. Rev. Biochem. 48, 327-386.

56. Janssens, W., Nkengasong, J., Heyndrickx, L., Van der Auwera, G., Vereecken, K., Coppens, S., Willems, B., Beirnaert, E., Fransen, K., Peeters, M. & van der Groen, G. (1999). Intrapatient variability of HIV type 1 group O ANT70 during a 10-year follow-up. AIDS Res. Hum. Retroviruses 15, 1325-1332.

57. Kusumi, K., Conway, B., Cunningham, S., Berson, A., Evan, C., Iversen, A. K., Colvin, D., Gallo, M. V., Coutre, S., Shpaer, E. G., Faulkner, D. V., deRonde, A., Volkman, S., Williams, C., Hirsch, M. S. & Mullins, J. I. (1992). Human immunodeficiency virus type 1 envelope gene structure and diversity in vivo after cocultivation in vitro. J. Virol. 66, 875-885.

58. Daniels, R. S., Smith, M. H. & Fisher, A. G. (1991). Molecular characterization of biologically diverse envelope variants of human immunodeficiency virus type 1 derived from an individual. J. Virol. 65, 5574-5578.

59. Fisher, A. G., Ensoli, B., Looney, D., Rose, A., Gallo, R. C., Saag, M. S., Shaw, G. M., Hahn, B. I. I. & Wong-Staal, F. (1988). Biologically diverse molecular variants within a single HIV-1 isolate. Nature 334, 444-447.

60. He, Y., Bowman, V. D., Mueller, S., Bator, C. M., Bella, J., Peng, X., Baker, T. S., Wimmer, E., Kuhn, R. J. & Rossmann, M. G. (2000). Interaction of poliovirus receptor with poliovirus. Proc. Natl. Acad. Sci. USA 97, 79-84.

61. Belnap, D. M., McDermott, B. M., Filman, D. J., Cheng, N., Trus, B. L., Zuccola, H. J., Racaniello, V. R., Hogle, J. M. & Steven, A. C. (2000). Three-dimensional structure of poliovirus receptor bound to poliovirus. Proc. Natl. Acad. Sci. USA 97, 73-78.

62. Kolatkar, P. R., Bella, J., Olson, N. H., Bator, C. M., Baker, T. S. & Rossmann, M. G. (1999). Structural studies of two rhinovirus serotypes complexed with fragments of their cellular receptor. EMBO J. 18, 6249-6259.

63. Rossman, M. G., Arnold, E., Erickson, J. W., Frankenberger, E. A., Griffith, J. P., Heckt, H. J., Johnson, J. E., Kamer, G., Luo, M., Mosser, A. G., Rueckert, R. R., Sherry, B. & Vriend, G. (1985). Structure of a human common cold virus and functional relationship to other picornaviruses. Nature 317, 145-153.

64. Hogle, J., Chow, M. & Filman, D. J. (1985). Three-dimensional structure of poliovirus at 2.9 ü resolution. Science 229, 1358-1365.

65. Mosser, A. G., Sgro, J. Y. & Rueckert, R. R. (1994). Distribution of drug resistance mutations in type 3 poliovirus identifies three reginos involved in uncoating functions. J. Virol. 68, 8193-8201.

66. Otwinowski, Z. & Minor, W. (1997). Processing of X-ray diffraction data collected in oscillation mode. Methods Enymol. 276, 307-326.

67. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T. & Warren, G. L. (1998). Crystallography & NMR system: A- new software suite for macromolecular structure determination. Acta Crystallogr. D Biol. Crystallogr. 54, 905-921.

68. Wu, L., Gerard, N. P., Wyatt, R., Choe, H., Parolin, C., Ruffing, N., Borsetti, A., Cardoso, A. A., Desjardin, E., Newman, W., Gerard, C. & Sodroski, J. (1996). CD4-induced interaction of primary HIV-1 gp120 glycoproteins with the chemokine receptor CCR-5. Nature 384, 179-183.

69. Lusty, C. J. (1999). A gentle vapor diffusion technique for cross-linking of protein crystals for cryocrystallography. J. Appl. Cryst. 32, 106-112.

70. Navaza, J. (1994). AMoRe: an automated package for molecular replacement. Acta Crystallogr. 50, 157-163.

71. Brunger, A. T. (1992). X-PLOR Version 3.1. A System for X-ray Crystallography and NMR. Yale University Press, New Haven, Conn.

72. Nicholls, A., Sharp, K. A. & Honig, B. (1991). Protein folding and association: insights from the interfacial and thermodynamic properties of hydrocarbons. Proteins Struct. Funct. Genet. 11, 281-296.

73. Kraulis, P. J. (1991). MOLSCRIPT: a program to produce both detailed and schematic plots of protein structures. J. Appl. Cryst. 24, 946-950.

74. Jones, T. A., Zou, J. Y., Cowan, S. W. & Kjeldgaard, M. (1991). Improved methods for building protein models in electron density maps and the location of errors in these models. Acta Crystallogr. A 47, 110-119.

75. Joseph-McCarthy D. et al. (1997) Use of the Multiple Copy Simultaneous Search (MCSS) Method to Design a New Class of Picornavirus Capsid Binding Drugs Struct. Funct. Genet. 29:32-58.

76. Grant, R. A. et al. (1994) Structures of poliovirus complexes with anti-viral drugs: implications for viral stability and drug desugn Curr Biol. September 1 4(9):784-797.

What is claimed is:

1. A method for identifying a compound which inhibits HIV-1 entry into a cell:
(a) determining the conformation of a crystal of the central cavity of HIV-1 gp120 in the presence of a ligand;
(b) contacting the crystal of HIV-1 gp120 with the compound;
(c) determining the conformation of the crystalized HIV-1 gp120 central cavity in the presence of the compound; and
(d) comparing the conformation of the crystalized HIV-1 gp120 central cavity determined in step (c) with the conformation of the crystalized HIV-1 gp120 in step (a) so as to identify a compound which inhibits HIV-1 entry into the cell.

2. The method of claim 1, wherein the ligand in step (a) is soluble CD4 or a soluble CD4 mimetic.

3. A compound identified by the method of claim 1.

4. A composition which comprises the compound of claim 3 and a carrier.

5. The composition of claim 4, wherein the carrier is a diluent, an aerosol, a topical carrier, an aqueous solution, a nonaqueous or a solid carrier.

6. A compound which inhibits the cavity binding interaction between HIV-1 gp120 and CD4.

7. A method of inhibiting HIV-1 infection of a cell comprising contacting the cell with an amount of the compound of claim 3 effective to inhibit HIV-1 infection, thereby inhibiting HIV-1 infection of the cell.

8. A method of preventing HIV-1 infection in a subject comprising administering to the subject an amount of the compound of claim 3 effective to inhibit HIV-1 infection, thereby preventing HIV-1 infection in the subject.

9. A method of treating HIV-1 infection in a subject comprising administering to the subject an amount of the compound of claim 3 effective to inhibit HIV-1 infection, thereby treating HIV-1 infection in the subject.

10. A method of inhibiting HIV-1 infection of a cell comprising contacting the cell with an amount of the compound of claim 6 effective to inhibit HIV-1 infection, thereby inhibiting HIV-1 infection of the cell.

11. A method of preventing HIV-1 infection in a subject comprising administering to the subject an amount of the compound of claim 6 effective to inhibit HIV-1 infection, thereby preventing HIV-1 infection in the subject.

12. A method of treating HIV-1 infection in a subject comprising administering to the subject an amount of the compound of claim 6 effective to inhibit HIV-1 infection, thereby treating HIV-1 infection in the subject.
